(19) United States
(12) Patent Application Publication
(10) Pub. No.: US 2014/0348897 A1
(43) Pub. Date: Nov. 27, 2014

(54) TREATMENT OR REDUCTION OF DENTAL CONDITIONS WITH ASCORBYL ESTERS

(71) Applicants: Dale S. Scherl, Lawrence, KS (US); Neil Ahle, Lawrence, KS (US); William D. Schoenherr, Hoyt, KS (US); Christopher Waldy, Maple Hill, KS (US)

(72) Inventors: Dale S. Scherl, Lawrence, KS (US); Neil Ahle, Lawrence, KS (US); William D. Schoenherr, Hoyt, KS (US); Christopher Waldy, Maple Hill, KS (US)

(73) Assignee: HILL'S PET NUTRITION, INC., Topeka, KS (US)

(21) Appl. No.: 14/365,902

(22) PCT Filed: Dec. 21, 2011
(86) PCT No.: PCT/US2011/066356

§ 371 Date: Jun. 16, 2014

(51) Int. Cl. A61K 8/49; A23K 1/00; A61K 8/92; A61K 8/02; A61Q 11/00; A61D 5/00; A23K 1/18

(52) U.S. Cl. CPC A61K 8/4973; A61D 5/00; A23K 1/00; A23K 1/18; A23K 1/1853; A61K 8/0204; A61Q 11/00; A61K 8/922; A61K 2800/622
USPC 424/442; 433/217.1; 424/49

(57) ABSTRACT

The present disclosure provides methods and compositions comprising ascorbyl esters for the treatment or reduction of dental conditions in an animal. A pet food composition of the disclosure can be a dry food composition comprising (i) a matrix of ingredients nutritionally or organoleptically adapted for the animal and (ii) an outer coating layer, wherein the outer coating layer is applied to the matrix as a solution comprising an effective amount of an ascorbic acid fatty acid ester dissolved in a plant oil. One disclosed method of treatment or reduction involves feeding an animal in need of such treatment a dry pet food composition of the disclosure. A second disclosed method comprises applying to one or more surfaces of one or more teeth of an animal in need of such treatment, an effective amount of a solution comprising an ascorbic acid fatty acid ester dissolved in a plant oil.

### FIELD

The present disclosure provides compositions comprising ascorbyl esters, including fatty acid esters, and the use thereof for the treatment or reduction of dental conditions in an animal. The disclosure provides dry pet food compositions comprising a coating layer comprising an effective amount of an ascorbyl ester, e.g., an ascorbyl fatty acid ester, dissolved in a solvent, e.g., a plant oil. The dental conditions may be treated or reduced by feeding the animal a composition of the disclosure and by direct application of a solution of the ascorbyl ester to the teeth of the animal in need thereof.

### BACKGROUND

Many animals, including domestic companion animals, e.g., canine and feline companion animals, accumulate dental plaque and calculus (tartar). These accumulations can lead to the animal's affliction with gingivitis, which, in turn, can progress to periodontitis that may eventually lead to even more serious systemic diseases and conditions. Animals afflicted with these dental conditions can also be generally afflicted with halitosis and stomatitis.

Dental plaque is formed as a result of the bacterial colonization of teeth and comprises a layer of bacteria on the erupted surfaces of teeth and in the gingival crevice. More specifically, dental plaque is a biofilm, usually a pale yellow that develops naturally on the teeth. Like any biofilm, dental plaque is formed by colonizing bacteria that attach themselves to a smooth surface such as the surface of a tooth. Plaque comprising specific bacterial species can lead to the development of dental caries and periodontal disease.

Dental calculus, or tartar, is a recurring calcified deposit on the surfaces of the teeth of many animals, including domesticated dogs and cats, as well as of on the teeth of humans, and primates. It is generally recognized that dental calculus develops in a sequential process that involves the accumulation of dental plaque and the subsequent calcification of the plaque by saliva, which contains high concentrations of calcium and phosphate.

Thus, dental calculus or tartar is a form of hardened dental plaque resulting from the continual accumulation of minerals from saliva on plaque that has developed on the teeth. The rough surface of calculus provides an ideal medium for further plaque formation, in somewhat autocatalytic manner that, if uninterrupted, can threaten gingival health.

Brushing and flossing can remove plaque from which calculus forms. Once calculus has been formed, however, it is generally too hard and firmly attached to be removed with a toothbrush. The accumulation of plaque and its sequential solidification or calcification as dental calculus or tartar eventually causes the gingiva to become irritated and inflamed, i.e., the condition referred to as gingivitis. Gingivitis in turn can progress and develop into periodontitis, which is characterized by a loss of the connective tissue fibers that attach the gums to the teeth and bone that surrounds the tooth. Calculus (tartar) is detrimental to gingival health because it serves as a substrate for increased plaque formation and retention.

Calculus deposits, once formed, can only be removed through dental prophylaxis or other mechanical procedures. Thus, the treatment of dental calculus is of importance not only for cosmetic reasons, but also because of their role in the development of gingivitis, stomatitis, periodontal disease, as well as the resultant potential for systemic infections, alveolar bone recession, interference in the normal mastication process, tooth loss and adverse mouth and breath odors.

Since the microorganisms making up dental plaque are recognized as the etiological agents responsible for the development of tartar and the subsequent inflammation of the oral soft tissues, it is well accepted that measures that inhibit or prevent formation of dental plaque will inhibit the progression from that condition to calculus accumulation, gingivitis, and ultimately to periodontitis.

However, compositions and methods that might be available for treatment of dental conditions in humans are not directly applicable for use in animals in light of, inter alia, the differences in compliance, cooperation, and temperament between animals, including companion canine and feline animals, and humans. Accordingly, a need exists for methods that can effectively control, reduce, or inhibit formation of dental plaque and calculus in animals. Additionally, a need exists for food products, compositions, solutions or methods that are useful for the effective control, reduction, or inhibition of formation of dental plaque and calculus in animals.

### SUMMARY

Provided herein is a pet food composition that is useful for treating or reducing a dental condition in an animal in need thereof. The pet food composition is a dry food composition comprising (i) a matrix of ingredients nutritionally or organoleptically adapted for the animal and (ii) an outer coating layer, wherein the outer coating layer is applied to the matrix as a solution comprising an effective amount of an ascorbyl ester dissolved in a solvent. In certain aspects of this embodiment, the ascorbyl ester is an ascorbic acid fatty acid ester. In other aspects of this embodiment, the solvent is an oil, e.g., a plant oil.

In one embodiment, the ascorbic acid is esterified with a fatty acid selected from the group consisting of lauric acid, myristic acid, palmitic acid, stearic acid, and arachidic acid. In a particular aspect of this embodiment, the ascorbic acid esterified with a fatty acid is palmitic acid.

In another embodiment, the ascorbyl ester of the outer coating layer is dissolved in a plant oil, which may be selected from among corn oil, cottonseed oil, canola oil, rapeseed oil, olive oil, palm oil, palm kernel oil, peanut oil, safflower oil, sesame oil, soybean oil, sunflower oil, wheat germ oil, almond oil, apricot oil, argan oil, avocado oil, ben oil, cashew oil, castor oil, grape seed oil, hazelnut oil, hemp seed oil, linseed oil, mustard oil, neem oil, pumpkin seed oil, rice bran oil, and combinations thereof. In a particular aspect of this embodiment, the solvent is soybean oil.

In further embodiments, the dental condition for which the composition is usefully administered is one or more of dental plaque accumulation, calculus, tooth staining, halitosis, stomatitis, gingivitis, and periodontitis. In one specific aspect of this embodiment, the dental condition is dental plaque accumulation. In another specific aspect of this embodiment, the dental condition is calculus.

In other embodiments, the matrix of the pet food composition further comprises an inner coating layer that is disposed between the matrix and the outer coating layer. The inner coating layer is applied to the surface of the matrix, and inhibits absorption of one or more components of the outer coating layer into the matrix.

In other embodiments, the outer coating layer is applied to the matrix in the form of a powder or “dusting,” while in still further embodiments, the solution of the ascorbyl ester in a solvent is encapsulated. In certain aspects of this embodiment, the encapsulated materials are applied to the surface of the matrix of ingredients of the dry pet food compositions. In another aspect, the ascorbyl ester solution is encapsulated and the resulting capsules are incorporated within the matrix of ingredients of the dry food composition.

Also provided herein are methods for treating or reducing a dental condition in an animal which comprise directly applying to the teeth of an animal in need thereof, an effective amount of a solution comprising an ascorbyl ester dissolved in a solvent. In certain aspects of this embodiment, the ascorbyl ester is an ascorbic acid fatty acid ester. In other aspects of this embodiment, the solvent is an oil, e.g., a suitable plant oil. In certain other aspects of this embodiment, the dental condition is selected from the group consisting of dental plaque accumulation, calculus, tooth staining, halitosis, stomatitis, gingivitis, periodontitis, and combinations thereof. In a specific embodiment, the methods disclosed herein are useful for treating or reducing dental plaque accumulation.

In one particular embodiment, the solution applied to the teeth comprises ascorbic acid esterified with a saturated fatty acid selected from among lauric acid, myristic acid, palmitic acid, stearic acid, and arachidic acid. In a particular aspect of this embodiment, the esterified derivative is ascorbyl palmitate.

In other specific aspects of this embodiment, the solvent used to dissolve the ascorbyl ester is an oil, e.g., a plant oil that can be any of corn oil, cottonseed oil, canola oil, rapeseed oil, olive oil, palm oil, palm kernel oil, peanut oil, safflower oil, sesame oil, soybean oil, sunflower oil, wheat germ oil, almond oil, apricot oil, argan oil, avocado oil, ben oil, cashew oil, castor oil, grape seed oil, hazelnut oil, hemp seed oil, linseed oil, mustard oil, neem oil, pumpkin seed oil, rice bran oil, and combinations thereof. In a particular aspect of this embodiment, the ascorbyl ester is an ascorbic acid fatty acid that is dissolved in soybean oil.

In another embodiment of the disclosure, ascorbyl esters and solutions thereof are encapsulated using materials and methods known in the art including but not limited to those disclosed in U.S. Pat. No. 4,895,72 and U.S. Patent Application Publication No. 2001/0195101 A1, each of which is hereby incorporated by reference in its entirety.

In another embodiment, the ascorbyl ester is applied to the surface of the matrix of ingredients, which may comprise an inner coating layer, in the form of a powder. Methods and materials for “dusting” the surface of a dry food composition with a composition are known in the art. Examples of suitable reagents, methods, and equipment include but are not limited to those disclosed in U.S. Patent Application Publication No. 20110027417 A1, which is hereby incorporated by reference in its entirety. In one illustrative embodiment of this method, a solution comprising an ascorbyl ester, e.g., an ascorbic acid fatty acid ester, is dissolved in a solvent, e.g., an oil and more specifically, a plant oil, mixed with other liquid topicals, e.g., a palatant, and loaded into a compressed air spray system and sprayed onto an uncoated base product, e.g., a kibble.

The methods of the present disclosure are useful for treating or reducing a dental condition in a companion animal, e.g., useful for treating or reducing a dental condition in a canine or feline.

In certain embodiments, a solution comprising an ascorbyl ester, e.g., an ascorbic acid fatty acid ester, dissolved in a solvent, e.g., an oil and more specifically, a plant oil, is applied directly to the teeth of the animal in need of such treatment using, e.g., a dental swab, a towelette, a sprayer, a sponge, or a brush. In a particular aspect of this embodiment, the solution can be applied with a device, e.g., a liquids applicator device that comprises an elongated hollow cartridge housing that contains a disposable, replaceable or refillable cartridge containing the solution. Such a device may further contain an applicator head portion that is attached to an open end of the cartridge housing. The device may also contain a dispensing mechanism that drives the solution from the cartridge to the applicator head. Contacting the teeth of the animal in need of treatment with the applicator head allows the user to coat or “paint” a tooth surface with the solution. One illustrative device for this purpose is that described in U.S. Pat. No. 7,607,852 B2, which is hereby incorporated by reference in its entirety.

Also provided herein is a method for treating or reducing a dental condition in an animal involving feeding an animal in need thereof a pet food composition of the disclosure, e.g., a dry pet food composition comprising (i) a matrix of ingredients nutritionally or organoleptically adapted for the animal and (ii) an outer coating layer that comprises an effective amount of an ascorbic acid fatty acid ester dissolved in an oil, e.g., a plant oil.

In one embodiment, the food composition comprises a second coating layer, i.e., an inner coating layer that is disposed between the matrix and the outer coating layer. The inner coating layer can, for example, be a coating that is applied to the surface of the matrix. The outer coating layer is then applied to the matrix that is already coated with the first coating layer. In one aspect of this embodiment, the inner coating layer serves to inhibit absorption of one or more components of the outer coating layer into the interior of the matrix.

In certain embodiments of this method, the dental condition treated or reduced is any of the following: dental plaque accumulation, calculus, tooth staining, halitosis, stomatitis, gingivitis, periodontitis, and combinations thereof. In a specific aspect of this embodiment, the dental condition treated or reduced is dental plaque accumulation.

In certain embodiments, the ascorbyl esters are derivatives of ascorbic acid that have been esterified with a suitable acid selected from among lauric acid, myristic acid, palmitic acid, stearic acid, and arachidic acid.

In one embodiment, the outer coating solution comprises a derivative of ascorbic acid in which ascorbic acid is esterified with a fatty acid selected from among lauric acid, myristic acid, palmitic acid, stearic acid, and arachidic acid, and, in a specific embodiment, the ascorbic acid is esterified with palmitic acid. In other aspects of this embodiment, the esterified ascorbic acid is dissolved in solvent such as but not limited to an oil, such as but not limited to a plant oil that is selected from among corn oil, cottonseed oil, canola oil, rapeseed oil, olive oil, palm oil, palm kernel oil, peanut oil, safflower oil, sesame oil, soybean oil, sunflower oil, wheat germ oil, almond oil, apricot oil, argan oil, avocado oil, ben oil, cashew oil, castor oil, grape seed oil, hazelnut oil, hemp seed oil, linseed oil, mustard oil, neem oil, pumpkin seed oil, rice bran oil, and combinations thereof. In a specific aspect of this embodiment, the esterified ascorbic acid is dissolved in soybean oil.

The pet food compositions of the present disclosure may be fed to a companion animal for treatment or reduction of a dental condition wherein that animal is a canine or feline. The present disclosure provides such compositions, e.g., pet food compositions, that are suitable for treating or reducing a dental condition in an animal in need thereof. In a specific embodiment, the composition is a particulate dry food composition, e.g. a “kibble” pet food, that comprises a matrix of ingredients nutritionally, organoleptically, or both nutritionally and organoleptically adapted for the animal and that comprise an outer coating layer applied to the surface of the matrix as a solution comprising an effective amount of an ascorbic acid fatty acid ester dissolved in an appropriate solvent. In one aspect of this embodiment, the solvent is an oil, such as but not limited to a plant oil.

In certain embodiments the matrix of the administered food comprises a second coating layer, i.e., an inner coating layer that is disposed between matrix and the outer coating layer. The inner coating layer can, for example, be a coating that is applied to the surface of the matrix. In this embodiment, the outer coating layer is applied to the matrix of ingredients that is already coated with the first coating layer. In one aspect of this embodiment, the inner coating layer serves to inhibit absorption of one or more components of the outer coating layer into the interior of the matrix.

In certain embodiments, the administered composition is a pet food composition in which the ascorbic acid is esterified with a fatty acid selected from among lauric acid, myristic acid, palmitic acid, stearic acid, and arachidic acid. In a particular aspect of this embodiment, the ascorbic acid is esterified with palmitic acid. In other aspects of this embodiment, the esterified ascorbic acid is dissolved in a plant oil selected from among corn oil, cottonseed oil, canola oil, rapeseed oil, olive oil, palm oil, palm kernel oil, peanut oil, safflower oil, sesame oil, soybean oil, sunflower oil, wheat germ oil, almond oil, apricot oil, argan oil, avocado oil, ben oil, cashew oil, castor oil, grape seed oil, hazelnut oil, hemp seed oil, linseed oil, mustard oil, neem oil, pumpkin seed oil, rice bran oil, and combinations thereof. In a specific aspect of this embodiment, the esterified ascorbic acid is dissolved in soybean oil.

These methods for treating or reducing a dental condition in an animal involving feeding a composition of the invention may be used for treating a dental condition in a companion animal, e.g., useful for treating or reducing a dental condition in a canine or a feline.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the illustrative and preferred embodiments of the disclosure are merely exemplary in nature and is in no way intended to limit the invention, its application, or uses.

### Definitions

The term “animal” as used herein refers to non-human animals, such as mammals and is particularly applicable to companion animals. Non-human mammals include non-human primates such as monkeys, chimpanzees, etc. Farm animals include goats, sheep, swine, cattle, etc. Wild and zoo animals include wolves, bears, deer, giraffes, elephants, etc. Non-mammalian animals include birds and working animals include horses.

The term “companion animal” used in the present invention includes any non-human animal suitable for being kept as a pet by humans including a dog, a cat, and a rodent. The compositions of the invention are in certain embodiments for the treatment of cats and/or dogs.

The term “dog” includes those dogs which are companion animals such as Canis familialis, working dogs and the like. The term dog is synonymous with the term canine

The term “cat” includes those cat, which are companion animals known as domestic cats or house cats. The term cat is synonymous with the term feline.

The term “treating,” in its various grammatical forms in relation to the present disclosure, refers to preventing, curing, reversing, attenuating, alleviating, ameliorating, minimizing, suppressing or halting the deleterious effects of one or more of the dental conditions disclosed herein.

The term “reducing,” in its various grammatical forms in relation to the present disclosure, e.g., “reducing a dental condition”, refers to lessening to a smaller extent, degree or intensity, in particular, lessening the dental condition to a smaller extent, degree or intensity, or lessening the symptoms associated with the dental condition to a smaller extent, degree or intensity.

As used herein, the terms “ascorbic acid fatty acid ester” and “ascorbyl fatty acid ester” are used to refer to ascorbic acid esterified to a fatty acid.

As used herein, “an amount effective,” “an effective amount,” and like terms refer to that amount of a compound, material or composition as described herein that may be effective to achieve a particular biological result, e.g., to treat or reduce a dental condition selected from the group consisting of dental plaque accumulation, calculus, tooth staining, gingivitis, periodontitis, stomatitis, halitosis, and combinations thereof. Such results may be achieved, for example, by feeding a pet food composition of the present disclosure to an animal in need of such treatment or by direct application of a solution of an ascorbyl ester in a solvent to one or more surfaces of one or more teeth of an animal in need of treatment of a dental condition. In specific embodiments, the esterified acid is a fatty acid, e.g., the ascorbyl ester is ascorbyl palmitate. In other specific aspects of this embodiment, the solvent is an oil such as but not limited to a plant oil. An effective amount may be based on several factors, including the particular animal's sex, age, weight, and the size of the animal's teeth as well as the animal's normal diet and its historical tendency to accumulate plaque.

As used herein, the term “coating” means a partial or complete covering, typically on the matrix of ingredients, that covers at least a portion of a surface, for example a surface of the matrix. In one example, the matrix may be partially covered with a coating such that only part of the matrix is covered, and part of the matrix is not covered and is thus exposed. In another example, the matrix may be completely covered with a coating such that the entire matrix is covered and thus not exposed. Therefore, a coating may cover from a negligible amount up to the entire surface. A coating, e.g., an outer coating layer, may be applied over another, e.g., an inner coating layer, resulting, for example, in a matrix comprising two layers of coating.

As used herein, the terms “dry food composition,” “pet food composition,” “dry pet food composition,” “companion animal pet food composition,” and “kibble,” all refer to a composition intended for ingestion by an animal, e.g., a companion canine or companion feline. Such foods may be formulated as nutritionally balanced compositions suitable for daily feed, and prepared as dry food compositions, e.g. kibbles, or they may be prepared as a dietary supplement or a treat, which may not necessarily be nutritionally balanced. As used herein, therefor, the term “kibble” includes a particulate pellet-like component of animal foods, such as dog and cat foods. Kibbles may range in texture from hard to soft and may have internal structures ranging from expanded structures to dense structures. Kibbles may be formed by an extrusion process.

As used herein, the phrase “matrix of ingredients” (“matrix”) includes but is not limited to the particulate pellets of a kibble. The matrix is typically formed from a mixture of ingredients. The matrix may be coated, with an outer coating layer or with both an inner coating layer and an outer coating layer, either or both of which may be a partial coating or a complete coating of the matrix. The matrix may comprise protein, carbohydrate, fat, and fiber, as well additional nutrients, vitamins, and minerals, and the like. In certain embodiments, the composition of the matrix is a nutritionally complete and nutritionally balanced composition. In certain aspects, the matrix of ingredients is nutritionally, organoleptically, or both nutritionally and organoleptically adapted for an animal.

The phrases “nutritionally balanced,” “nutritionally complete,” and “nutritionally adapted,” as used herein, refer to compositions, e.g., pet food compositions, that comprise the known required nutrients to sustain life in the proper amounts and proportions, where those amounts and proportions are based upon the recommendations of recognized authorities, including governmental agencies, such as, but not limited to the United States Food and Drug Administration's Center for Veterinarian Medicine, and the Association of American Feed Control Officials Incorporated, in the field of pet nutrition, except for the additional need for water.

The present disclosure also provides compositions useful for treating or reducing a dental condition in an animal in need of such treatment. In one aspect of this embodiment, the composition is a dry pet food composition, and more specifically, it is a dry food composition comprising (i) a matrix of ingredients nutritionally, organoleptically or nutritionally and organoleptically adapted for the animal, and (ii) an outer coating layer, wherein the outer coating layer is applied to the matrix as a solution comprising an effective amount of an ascorbyl ester, e.g., an ascorbic acid fatty acid ester dissolved in solvent, e.g., an oil, such as a plant oil. In one aspect of this embodiment, the matrix of ingredients further comprises an inner coating layer disposed between the matrix and the outer coating layer, in which the inner coating layer is applied to the surface of the matrix, and wherein the inner coating layer inhibits absorption of one or more components of the outer coating layer into the matrix.

In one embodiment, the dry food composition comprising a matrix to be coated with the outer coating layer that comprises an ascorbic acid fatty acid ester dissolved in a plant oil is formulated to meet the ordinary nutritional requirements of the animal to which the methods disclosed herein are to be applied. Such formulations can be determined by a skilled artisan based upon the animal's species, age, sex, weight, and other factors.

In specific embodiments, compositions of the disclosure comprise a matrix of ingredients formulated with suitable sources of protein, carbohydrate, fat, and fiber before coating.

The pet food compositions of the disclosure include protein. Suitable sources of protein are known to those in the art and may be obtained, for example, from a variety sources such as plants, animals, or both. Animal protein includes meat, meat by-products, dairy, and eggs. Meats include the flesh from poultry, fish, and animals such as cattle, swine, sheep, goats, and the like, meat by-products including lungs, kidneys, brain, livers, stomachs, and intestines. In certain embodiments, the protein of the pet food compositions of the disclosure can include meat, a meat by-product, dairy products, eggs, and combinations thereof. The pet food compositions of the disclosure may also comprise free amino acids, peptides, and combinations thereof

The pet food compositions of the disclosure may comprise one or more sources of fat and carbohydrate, including those known to those in the art. Illustrative examples of suitable sources of carbohydrate and fat include, but are not to be limited to, animal fat, fish oil, vegetable oil, meat, meat by-products, grains, other animal or plant sources, and mixtures thereof Grains include wheat, sorghum, corn, barley, rice and the like.

The pet food compositions of the disclosure may also comprise one or more sources of fiber, which may be classified as soluble or insoluble fiber. Illustrative but non-limiting sources of fiber include wherein the fiber food ingredient is obtained from a variety of sources such as vegetable fiber sources, for example, cellulose, beet pulp, peanut hulls, and soy fiber.

In certain embodiments, the pet food compositions of the disclosure may also comprise nutritional balancing agents that are obtained from a variety of sources known to skilled artisans, for example, vitamin and mineral supplements and food ingredients. Vitamins and minerals can be included in amounts required to avoid deficiency and maintain health. These amounts are readily available in the art. The Association of American Feed Control Officials (AAFCO) provides recommended amounts of such nutrients for dogs and cats. See Association of American Feed Control Officials, Inc., Official publication, pp. 159-162 (2011). Vitamins generally useful as food additives include vitamin A, vitamin B₁, vitamin B₂, vitamin B₆, vitamin B₁₂, vitamin D, biotin, vitamin K, folic acid, inositol, niacin, and pantothenic acid. Minerals and trace elements useful as food additives include calcium, phosphorus, sodium, potassium, magnesium, copper, zinc, chloride, iron, selenium, iodine, and iron.

In certain other embodiments, the pet food compositions of the disclosure may contain other ingredients, in addition to vitamins and minerals, such as fillers, palatability enhancers, binding agents, flavors, stabilizers, emulsifiers, sweeteners, colorants, buffers, salts, coatings, and the like known to skilled artisans. Stabilizers include substances that tend to increase the shelf life of the composition such as preservatives, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Specific amounts for each composition component, food ingredient, and other ingredients will depend on a variety of factors such as the particular components and ingredients included in the composition; the species of animal; the animal's age, body weight, general health, sex, and diet; the animal's consumption rate; the type of disease or condition being treated; and the like. Therefore, the component and ingredient amounts may vary widely and may deviate from the preferred proportions described herein.

In another aspect of this embodiment, the companion animal pet food composition comprises a matrix of ingredients that, before coating, comprises from about 10% to about 35%, from about 15% to about 30%, and from about 20% to about 25% protein. In a specific aspect, the companion animal pet food composition comprises a matrix of ingredients that, before coating, comprises about 22% protein.

In still another aspect of this embodiment, companion animal pet food composition comprises a matrix of ingredients that, before coating, comprises from about 35% to about 75%, from about 45% to about 70%, and from about 55% to about 65% carbohydrate. In a specific aspect, the composition is a companion animal pet food composition in which the matrix of ingredients, before coating, comprises about 60% carbohydrate.

In still another aspect of this embodiment, the companion animal pet food composition comprises a matrix of ingredients that, before coating, comprises from about 3% to about 25%, from about 5% to about 20%, from about 10% to about 15%, and from about 8% to about 14% fat. In a specific aspect, the composition of the disclosure is a companion animal pet food composition containing a matrix of ingredients that, before coating, comprises about 10% fat.

In another aspect of this embodiment, the companion animal pet food composition comprises a matrix of ingredients that, before coating, comprises from about 0.25% to about 10%, from about 0.5% to about 6%, from about 1% to about 5% crude fiber, and from about 2% to about 4% crude fiber. In a specific aspect, the composition is a companion animal pet food composition comprising a matrix of ingredients that, before coating, comprises about 3% crude fiber.

In a further aspect of this embodiment, the companion animal pet food composition comprises a matrix of ingredients that, before coating, comprises from about 5% to about 15%, from about 6% to about 14%, from about 7% to about 13%, from about 8% to about 12%, and from about 9% to about 11% moisture. In a specific aspect, the composition comprises a matrix of ingredients that, before coating, comprises about 10% moisture.

In a particular aspect of this embodiment, the composition is a companion animal pet food composition comprising a matrix of ingredients that, before coating, comprises from about 20% protein, about 60% carbohydrate, about 13% fat, about 10% moisture and from about 2.5% to about 8.5% crude fiber.

The pet food compositions comprising a matrix of ingredients formulated as above are particularly suitable for feeding to canines in need of dental treatment or reduction in dental conditions. In other embodiments, pet food compositions comprising a matrix of ingredients particularly suitable for use in the disclosed methods for feline companion animals generally have higher levels of protein and lower levels of carbohydrate and fiber than described above.

Therefore, in another embodiment, the disclosure provides a composition comprising a matrix of ingredients that, before coating, comprises from about 20% to about 45%, from about 25% to about 40%, and from about 30% to about 35% protein. In a specific aspect, the composition is companion animal dry pet food composition, primarily intended for but not limited to felines, comprising a matrix of ingredients that, before coating, comprises about 33% protein.

In a particular aspect of this embodiment, the composition comprises a matrix of ingredients that, before coating, comprises from about 15% to about 45%, from about 20% to about 40%, and from about 25% to about 35% carbohydrate. In a specific aspect, the composition is a companion animal dry pet food composition, primarily intended for but not limited to felines, comprising a matrix of ingredients that, before coating, comprises about 30% carbohydrate.

In still another embodiment, the composition comprises a matrix of ingredients that, before coating, comprises from about 5% to about 35%, from about 10% to about 30%, and from about 15% to about 25% fat. In a specific aspect, the composition is a companion animal dry pet food composition, primarily intended for but not limited to felines, that comprises a matrix of ingredients that, before coating, comprises about 20% fat.

In another aspect of this embodiment, the composition is a companion animal pet food composition in which the matrix of ingredients comprises from about 4% to about 15%, from about 5% to about 12%, from about 6% to about 10%, and from about 7% to about 9% crude fiber. In a specific aspect, the companion animal pet food composition comprises a matrix of ingredients that, before coating, comprises about 8% crude fiber.

In another aspect of this embodiment, the composition is a companion animal dry pet food composition, primarily intended for but not limited to felines, in which the matrix of ingredients comprises about 33% protein, about 30% carbohydrate, about 20% fat, about 8% crude fiber and about 10% moisture.

In one illustrative example, the matrix of ingredients of the composition, before coating, may comprise inter alia, vitamins, minerals and other additives. In one aspect of this embodiment, the matrix of ingredients of an illustrative pet food may comprise, before coating, chicken corn meal, ground grain sorghum, ground wheat, chicken by-product meal, brewers rice, soybean meal, animal fat, (preserved with BHA and propyl gallate), natural flavor, vegetable oil, dried egg product, flaxseed, preserved with BHT and BHA, beta-carotene, minerals (iodized salt, calcium carbonate, dicalcium phosphate, potassium chloride, ferrous sulfate, zinc oxide, copper sulfate, manganous oxide, calcium iodate, sodium selenite), and vitamins (choline chloride, vitamin A supplement, vitamin D₃ supplement, vitamin E supplement, niacin, thiamine, calcium pantothenate, pyridoxine hydrochloride, riboflavin, folic acid, biotin, vitamin B₁₂ supplement).

In particular embodiments, the matrix of ingredients of the compositions of the disclosure are prepared in a dry form using conventional processes. In one contemplated embodiment, dry ingredients, including, for example, animal protein sources, plant protein sources, grains, etc., are ground and mixed together. Moist or liquid ingredients, including fats, oils, animal protein sources, water, etc., are then added to and mixed with the dry mix. The mixture is then processed into kibbles or similar dry pieces. Kibble is often formed using an extrusion process in which the mixture of dry and wet ingredients is subjected to mechanical work at a high pressure and temperature, and forced through small openings and cut off into kibble by a rotating knife. The wet kibble is then dried and optionally coated, e.g., with an inner coating layer, before coating with the solution of an ascorbic acid fatty acid ester in a plant oil. Kibble also can be made from the dough using a baking process, rather than extrusion, wherein the dough is placed into a mold before dry-heat processing. Kibble also can be made from a food matrix undergoing pelletization.

In another embodiment, the matrix of ingredients that is optionally coated with an inner coating layer before coating with the solution of an ascorbic acid fatty acid ester in a plant oil is prepared as a food supplement. Supplements include, for example, a feed or pet food used with another feed or pet food to improve the nutritive balance or performance of the total. The contemplated supplements include compositions that are fed undiluted as a supplement to other feeds or pet foods, offered free choice with other parts of an animal's ration that are separately available, or diluted and mixed with an animal's regular feed or pet food to produce a complete feed or pet food.

Compositions of the present disclosure may be formulated and prepared as treats. Treats of the present disclosure can be prepared by, for example, an extrusion or baking process similar to those described above for dry food to provide the dry compositions that are then optionally coated with an inner coating layer, before coating with the solution of an ascorbic acid fatty acid ester in a plant oil. Therefore, in another embodiment, the composition that is optionally coated with an inner coating layer, before coating with the solution of an ascorbic acid fatty acid ester in a plant oil, is prepared as a treat. Treats include, for example, compositions that are given to an animal to entice the animal to eat during a non-meal time. Contemplated treats for canines include, for example, dog biscuits in the shape of dog bones. Treats can be nutritional, wherein the composition comprises one or more nutrients, and can, for example, have a composition as described above for food. Non-nutritional treats encompass any other treats that are non-toxic. In each instance, the dry material is coated with the solution of an ascorbic acid fatty acid ester in a plant oil. In certain aspects, the treat further comprises an optional inner coating layer disposed between the outer surface of the treat and the outer coating layer of the solution of an ascorbic acid fatty acid ester in a plant oil.

In certain embodiments, the matrix of ingredients of the compositions used in the methods of the present disclosure, whether a companion animal pet food composition, dietary supplement, or treat may further comprise at least one of a commercial liquid palatant enhancer or other flavor composition to create a novel flavor palatant which can then be included in the formulation of the composition. Suitable commercial liquid palatant enhancers for use with compositions used in the disclosed methods can include any known or commercially available liquid palatant enhancers commercially available from pet food palatant enhancers or other flavor suppliers known to those of skill in the art.

The compositions of the disclosure comprise physically discrete particles which include kibbles, as well as, in other embodiments, bits and other discrete materials, such as a solid treat, a supplement or the like, which are coated with an outer coating layer applied as a solution of ascorbic acid fatty acid ester in a plant oil, and which may further comprise an inner coating layer disposed on the surface of the matrix of ingredients before application of the outer coating layer. Again, by coating the surface is meant that at least enough of the surface of the matrix of ingredients is covered with the film so as to achieve the desired effect. The entire surface need not be coated. For example, in certain embodiments, an effective amount of the ascorbic acid fatty acid ester can be delivered by feeding dry food compositions comprising a matrix of ingredients in which about 5%, about 10%, about 15%, about 20%, about 25%, about 30%, about 40%, about 45%, about 50%, about 60%, about 70%, about 80%, about 90%, or up to about 100% of the surface of the matrix has been coated with either or both of the outer coating layer and, when present, the inner coating layer.

The processes by which the outer coating layer, as well as the inner coating layer (when present) is applied to the matrix of ingredients (or the matrix of ingredients carrying the inner coating layer) employ any of the common procedures known to be effective in applying films to objects. These procedures include casting, spraying, grafting, sputtering, flowing, calendaring, and the like, and can be employed with the matrix of ingredients prior to being cut into physically discrete portions or, preferably, after the matrix of ingredients is formed, thus ensuring an even distribution on the entire matrix, if so desired.

In certain embodiments, the inner coating layer comprises a polymer which can be any of zein, casein, starch, cellulose, gum, gelatin, starch/synthetic polymer, e.g., starch/low density polyethylene, and the like, as well as combinations thereof. In certain aspects of this embodiment, the inner coating layer can be from about 1 to about 2000 microns thick, or from about 2 to about 1000 microns. The composition and thickness of the inner coating layer are chosen to provide the desired degree of inhibition of absorption of one or more components of the outer coating layer into the matrix of ingredients.

In certain embodiments, an aqueous solution, sol, or suspension that is applied to the surface of the matrix of ingredients to form the inner coating layer may comprise monosaccharides such as glucose, fructose, mannose, arabinose; disaccharides and trisaccharides such as sucrose, lactose, maltose, trehalose, lactulose; corn and rice syrup solids; dextrins such a corn, wheat, rice and tapioca dextrins; maltodextrins; starches such as rice, wheat, corn, potato, tapioca starches, or these starches modified by chemical modification. In other aspects, the aqueous solution, sol, or suspension that is applied to the surface of the matrix of ingredients to form the inner coating layer may comprise oligosaccharides such as fructooligosccharides, alginates, chitosans; gums such as carrageen, and gum arabic; polyols such as glycerol, sorbitol, mannitol, xylitol, erythritol; esters of polyols such as sucrose esters, polyglycol esters, glycerol esters, polyglycerol esters, sorbitan esters; sorbitol; molasses; honey; gelatins; peptides; proteins and modified proteins such as whey liquid, whey powder, whey concentrate, whey isolate, whey protein isolate, or a high lactose whey by-product. In other aspects of this embodiment, the aqueous solution, sol, or suspension that is applied to the surface of the matrix of ingredients to form the inner coating layer may comprise meat broth solids such as chicken broth, chicken broth solids, soy protein, and egg white.

In further aspects of this embodiment, the aqueous solution, sol, or suspension that is applied to the surface of the matrix of ingredients to form the inner coating layer may comprise a palatant. Palatants useful for this purpose are palatability enhancers which are typically employed to enhance the overall palatability of the composition and potentially to overcome any negative flavor effects arising from other components of the composition. Palatants particularly useful in accordance with the invention are those described in US2003/0086961, which is hereby incorporated by reference. Particularly useful are protein hydrolysates which typically provide a savory taste attractive to various animals including cats and dogs. The palatants include animal digest; animal hydrolysates; animal internal organs such as liver, lungs and heart; meats such as lamb, beef, pork, chicken and turkey; seafoods such as fish, crab, and shrimp; dairy products such as milk and cheese; yeasts; peptides; amino acids; nucleotides; fat; oil; artificial meat and/or seafood flavors; mallard reactants; sugars; plant extracts and other aromas nature and/or artificial that are attractive to the animals. For cats, preferred palatants include chicken liver hydrolysates and fish-based hydrolysates; for dogs, a protein digest or hydrolysate is particularly useful.

In further aspects of this embodiment, the aqueous solution, sol, or suspension that is applied to the surface of the matrix of ingredients to form the inner coating layer may comprise combinations of two or more of any of these materials.

In certain embodiments therefore, the inner coating layer may be prepared as an aqueous solution, sol, or suspension that is applied to the surface of the matrix of ingredients using any of the methods disclosed above as well as any known in the art using, e.g., commercially-available equipment. In certain aspects of these embodiments the amount of aqueous material applied to the matrix of ingredients represents from about 1% to about 20%, from about 2% to about 15%, or from about 5 to about 10% of the weight of the matrix of ingredients. The coated matrix is, generally, then dried before application of the outer coating layer. In one aspect of this embodiment, the matrix of ingredients to which the inner coating layer has been applied are placed in a tumbling drum to facilitate uniform distribution of the coating layer. In certain instances the matrix of ingredients in the tumbling drum can be exposed to heated air for drying, e.g., reducing the moisture content of the coated matrix of ingredients to a level of about 10%. Accordingly, after drying, the inner coating layer represents from about 0.025% to about 10%, from about 0.05% to about 7.5%, from about 0.1% to about 5.0%, from about 0.2% to about 2.5%, or from about 0.25% to about 1.25% of the weight of the matrix of ingredients on a dry matter basis.

In one illustrative, non-limiting embodiment, the inner coating layer is formulated to comprise a fat (e.g., choice white grease), a palatant (e.g., an acid hydrolysate of chicken liver), an oil (e.g., soybean oil), and a fat-soluble antioxidant or vitamin (e.g., vitamin E).

The inner coating layer, in certain embodiments inhibits absorption of one or more components of the outer coating layer into the matrix of ingredients, thereby maximizing the amount of ascorbic fatty acid ester and plant oil solvent disposed on the surface of the coated matrix.

The outer coating layer is prepared by dissolving the ascorbyl ester, e.g., an ascorbic acid fatty acid ester, in a solvent, e.g., a plant oil, and applying that solution to the matrix of ingredients that, in certain embodiments, may already comprise an inner coating layer. The specific combination of fatty acid for esterification of ascorbic acid and the plant oil solvent used will depend on the solubility of the former in the latter, the acceptability of the taste of the solution to the animal to be treated, as well as the residence time of the solution on the teeth of the treated animal. Each of these parameters can be readily determined.

In one aspect of this embodiment, the solvent is a plant oil that can be selected from among corn oil, cottonseed oil, canola oil, rapeseed oil, olive oil, palm oil, palm kernel oil, peanut oil, safflower oil, sesame oil, soybean oil, sunflower oil, wheat germ oil, almond oil, apricot oil, argan oil, avocado oil, ben oil, cashew oil, castor oil, grape seed oil, hazelnut oil, hemp seed oil, linseed oil, mustard oil, neem oil, pumpkin seed oil, rice bran oil, and combinations thereof. In a specific aspect, the plant oil is soybean oil.

In another aspect of this embodiment, the fatty acid that is esterified to ascorbic acid is any of lauric acid, myristic acid, palmitic acid, stearic acid, and arachidic acid. In a specific aspect, the esterified fatty acid is palmitic acid, i.e., the ascorbic acid fatty acid ester is ascorbyl palmitate.

In certain embodiments, the outer coating layer comprises an ascorbic acid fatty acid ester dissolved in a plant oil. In another embodiment, the outer coating layer consists essentially of an ascorbic acid fatty acid ester dissolved in a plant oil, and in a specific aspect of this embodiment, the ascorbic acid fatty acid ester is the only active agent in the solution applied to the matrix of ingredients to form the outer coating layer. In a still further embodiment, the outer coating layer consists of an ascorbic acid fatty acid ester dissolved in a plant oil.

In a specific embodiment, the outer coating layer comprises ascorbyl palmitate dissolved in soybean oil. In another embodiment, the outer coating layer consists essentially of ascorbyl palmitate dissolved in soybean oil and, in specific aspect of this embodiment, ascorbyl palmitate is the only active agent in the solution applied to the matrix of ingredients to form the outer coating layer. In a still further embodiment, the outer coating layer consists of ascorbyl palmitate dissolved in soybean oil.

The process by which the outer coating layer is applied to the matrix of ingredients (or the matrix of ingredients carrying the inner coating layer) may employ any of the common procedures known to be effective in applying films to objects as well as commercially-available equipment useful for such purposes. These procedures include, but are not limited to, casting, spraying, grafting, sputtering, flowing, calendaring, and the like. Application of the outer coating layer to the matrix of ingredients may be carried out before the formulation, e.g., the cooked and extruded material, is cut into physically discrete portions or, preferably, after the physically discrete particles of the matrix of ingredients are formed, thus ensuring an even distribution over the entire matrix. In one aspect of this embodiment, the matrix of ingredients to which the outer coating layer has been applied is placed in a tumbling drum to facilitate uniform distribution of the solution of the outer coating layer over the matrix.

In one embodiment, the outer coating layer represents from about from about 0.025% to about 10%, from about 0.05% to about 7.5%, from about 0.1% to about 5.0%, from about 0.2% to about 2.5%, or from about 0.25% to about 1.25% of the weight of the matrix of ingredients on a (w/w) basis. Accordingly, the ascorbic acid fatty acid ester of the outer coating layer can represent 0.005% to about 5%, from about 0.010% to about 2.5%, from about 0.025% to about 1.0%, from about 0.050% to about 0.5%, of the weight of the coated matrix of ingredients, on a dry matter basis. In a specific aspect of this embodiment, the ascorbic acid fatty acid ester is ascorbyl palmitate and it represents about 0.1% of the weight of the matrix of ingredients on a dry matter basis.

In certain embodiments, the outer coating layer is prepared by dissolving the ascorbyl ester, e.g., an ascorbic acid fatty acid ester, in a solvent, e.g., a plant oil, and mixing that solution with an aqueous solution, sol, or suspension, and applying that solution to the matrix of ingredients. The specific combination of fatty acid for esterification of ascorbic acid and the plant oil solvent used will depend on the solubility of the former in the latter, the acceptability of the taste of the solution to the animal to be treated, as well as the residence time of the solution on the teeth of the treated animal. Each of these parameters can be readily determined.

The aqueous solution, sol, or suspension that is mixed in with the ascorbyl ester dissolved in a solvent may comprise monosaccharides such as glucose, fructose, mannose, arabinose; disaccharides and trisaccharides such as sucrose, lactose, maltose, trehalose, lactulose; corn and rice syrup solids; dextrins such a corn, wheat, rice and tapioca dextrins; maltodextrins; starches such as rice, wheat, corn, potato, tapioca starches, or these starches modified by chemical modification. In other aspects, the aqueous solution, sol, or suspension that is that is mixed in with the ascorbyl ester dissolved in a solvent may comprise oligosaccharides such as fructooligosccharides, alginates, chitosans; gums such as carrageen, and gum arabic; polyols such as glycerol, sorbitol, mannitol, xylitol, erythritol; esters of polyols such as sucrose esters, polyglycol esters, glycerol esters, polyglycerol esters, sorbitan esters; sorbitol; molasses; honey; gelatins; peptides; proteins and modified proteins such as whey liquid, whey powder, whey concentrate, whey isolate, whey protein isolate, or a high lactose whey by-product. In other aspects of this embodiment, the aqueous solution, sol, or suspension that is mixed in with the ascorbyl ester dissolved in a solvent may comprise meat broth solids such as chicken broth, chicken broth solids, soy protein, and egg white.

In further aspects of this embodiment, the aqueous solution, sol, or suspension that is mixed in with the ascorbyl ester dissolved in a solvent may comprise a palatant. Palatants useful for this purpose are palatability enhancers which are typically employed to enhance the overall palatability of the composition and potentially to overcome any negative flavor effects arising from other components of the composition. Palatants particularly useful in accordance with the invention are those described in US2003/0086961, which is hereby incorporated by reference. Particularly useful are protein hydrolysates which typically provide a savory taste attractive to various animals including cats and dogs. The palatants include animal digest; animal hydrolysates; animal internal organs such as liver, lungs and heart; meats such as lamb, beef, pork, chicken and turkey; seafoods such as fish, crab, and shrimp; dairy products such as milk and cheese; yeasts; peptides; amino acids; nucleotides; fat; oil; artificial meat and/or seafood flavors; mallard reactants; sugars; plant extracts and other aromas nature and/or artificial that are attractive to the animals. For cats, preferred palatants include chicken liver hydrolysates and fish-based hydrolysates; for dogs, a protein digest or hydrolysate is particularly useful.

In further aspects of this embodiment, the aqueous solution, sol, or suspension that is mixed in with the ascorbyl ester dissolved in a solvent may comprise combinations of two or more of any of these materials.

In certain embodiments therefore, the aqueous solution, sol, or suspension that is mixed in with the ascorbyl ester dissolved in a solvent may be prepared using any of the methods disclosed above as well as any known in the art using, e.g., commercially-available equipment. In certain aspects of these embodiments the amount of aqueous material applied to the matrix of ingredients represents from about 1% to about 20%, from about 2% to about 15%, or from about 5 to about 10% of the weight of the matrix of ingredients.

In one illustrative, non-limiting embodiment, the aqueous solution, sol, or suspension that is mixed in with the ascorbyl ester dissolved in a solvent is formulated to comprise a fat (e.g., choice white grease), a palatant (e.g., an acid hydrolysate of chicken liver), an oil (e.g., soybean oil), and a fat-soluble antioxidant or vitamin (e.g., vitamin E).

The present disclosure therefore provides compositions useful for treating or reducing a dental condition in an animal in need thereof. In one aspect of this embodiment, the composition is a pet food composition, and more specifically, it is a particulate dry food composition comprising matrix of ingredients and an outer coating layer, wherein the outer coating layer is applied to the matrix of ingredients as a solution comprising an effective amount of an ascorbic acid fatty acid ester dissolved in a plant oil. In one aspect of this embodiment, the matrix of ingredients further comprises an inner coating layer disposed between the matrix and the outer coating layer, in which the inner coating layer is applied to the surface of the matrix of ingredients, and wherein the inner coating layer inhibits absorption of at least one component of the outer coating layer into the matrix.

The present disclosure also provides methods and compositions for treating or reducing a dental condition in an animal either by direct application of a solution comprising an ascorbyl ester in a solvent to one or more surfaces of one or more teeth of an animal in need thereof. In certain aspects of this embodiment, the ascorbyl ester is an ascorbic acid fatty acid ester, e.g., ascorbyl palmitate. In other aspects the solvent is an oil, e.g., a plant oil such as but not limited to soybean oil.

In one embodiment, the present disclosure provides a method for treating or reducing a dental condition in an animal that comprises applying an effective amount of a solution comprising an ascorbic acid ester dissolved in a solvent oil to one or more surfaces of one or more teeth of an animal in need thereof. In certain aspects, the ascorbyl ester is an ascorbyl fatty acid ester. In certain aspects the solvent is an oil, such as but not limited to a plant oil. In certain embodiments, the dental condition to be treated or reduced is any of plaque accumulation, 1 accumulation, tooth staining, halitosis, stomatitis, gingivitis, periodontitis, and combinations thereof. In a particular aspect of this embodiment, the dental condition to be treated or reduced is plaque accumulation.

Plaque treatment or reduction is important since this condition is the first step in a progression of developing oral conditions including tartar accumulation, tooth staining, halitosis, stomatitis, gingivitis, periodontitis. Therefore, the presently disclosed methods and compositions that are useful for treating or reducing plaque will also be useful for treating (as that term is defined herein) or reducing, tartar accumulation, tooth staining, halitosis, stomatitis, gingivitis, periodontitis, and combinations thereof.

In particular embodiments, the solution can be applied e.g., on a daily basis, or on a weekly basis, or as needed and may be continued on such bases as required or desired to treat or reduce plaque developing on the teeth of the animal in need thereof.

As noted above, the ascorbyl ester can be an ascorbic acid fatty acid ester. Ascorbyl fatty acid esters useful in the methods and compositions disclosed herein may be prepared from any effective fatty acid including unsaturated and saturated fatty acids having a carbon chain of 8, 10, 12, 14, 16, 18, 20, 22, 24, or 26 carbons. In certain aspects of this embodiment, the fatty acid and the ascorbyl fatty acid ester prepared therefrom are generally recognized as safe (GRAS) compounds and food ingredients. The fatty acid to be esterified can be obtained by hydrolysis of naturally-occurring triglycerides and phospholipids.

In certain aspects of this embodiment, the esterified fatty acid is selected from the group consisting of lauric acid, myristic acid, palmitic acid, stearic acid, and arachidic acid. In one specific aspect of this embodiment, the esterified fatty acid is palmitic acid and, therefore the fatty acid ester of the applied solution is ascorbyl palmitate.

As noted above, the solvent for the ascorbyl ester can be an oil such as but not limited to a plant oil. Accordingly, in certain embodiments, the solvent used to dissolve the ascorbyl ester is a plant oil selected from any of corn oil, cottonseed oil, canola oil, rapeseed oil, olive oil, palm oil, palm kernel oil, peanut oil, safflower oil, sesame oil, soybean oil, sunflower oil, wheat germ oil, almond oil, apricot oil, argan oil, avocado oil, ben oil, cashew oil, castor oil, grape seed oil, hazelnut oil, hemp seed oil, linseed oil, mustard oil, neem oil, pumpkin seed oil, rice bran oil, and combinations thereof. In particular aspects of this embodiment, the plant oil is one that is generally recognized as safe (GRAS), e.g., for use in compositions applied to the skin or teeth of an animal and for use as a food ingredient that will be consumed by non-human animals. In a specific aspect of this embodiment, the plant oil solvent for the ascorbyl ester is soybean oil.

In certain embodiment, the ascorbic acid fatty acid ester is ascorbyl palmitate and the solvent is soybean oil. This solution may comprise from about 5% to about 50%, from about 10% to about 40%, from about 15% to about 30%, or from about 20% to about 25% (w/w) ascorbyl palmitate in the solution. In a specific aspect, the applied solution comprises ascorbyl palmitate dissolved in soybean oil to a concentration of about 23% (w/w) (weight ascorbyl palmitate/weight of solution).

In certain embodiments, the specific combination of fatty acid for esterification of ascorbic acid and the plant oil solvent used will depend on the solubility of the former in the latter, the acceptability of the taste of the solution to the animal to be treated, as well as the residence time of the solution on the teeth of the treated animal. Each of these parameters can be readily determined and compared to the specific combination of ascorbyl palmitate dissolved in soybean oil.

In one embodiment, the disclosed methods are used for treatment or reduction of a dental condition in an animal in need thereof, where the animal is a companion animal. In a particular aspect of this embodiment, the animal is a canine while in another aspect, the animal is a feline.

In certain non-limiting embodiments, the solution of ascorbic acid fatty acid ester in plant oil is directly applied to one or more surfaces of one or more teeth of an animal in need of treatment for a dental condition using, e.g., a dental swab, a towelette, a sprayer, a sponge, or a brush. In one illustrative aspect of this embodiment, the solution is provided in a container with a threaded cap having a brush attached to the inside, unthreaded surface of the cap. In other aspects of this embodiment the dental swab, towelette, sprayer, or sponge is pre-loaded with an effective dose of the solution of the ascorbic acid fatty acid ester in plant oil. In certain aspects, each of the dental swab, towelette, sprayer, or sponge is intended for a single use and each is individually packed, e.g., in a sealed wrapper.

In another particular embodiment, the solution of the ascorbic acid fatty acid ester in plant oil is provided in a cartridge for use in a liquids applicator device. In one aspect the cartridge contains a plurality of effective doses. In another aspect the cartridge contains a single effective dose of the solution of the ascorbic acid fatty acid ester in plant oil, i.e., an amount sufficient for application to all of the surfaces of all of the teeth of the animal in need of treatment. According to this embodiment, the ascorbic acid fatty acid ester solution in plant oil is applied with a liquids applicator device that comprises an elongated hollow cartridge housing. The cartridge is inserted within the housing and connected to an applicator head portion attached to an open end of the cartridge housing. The head portion is removable and, in one aspect, comprise a sponge or pad attached to the end thereof. The device also can contain a liquids dispensing mechanism that causes the solution to be dispensed from the cartridge into the applicator head portion, which is contacted with the tooth surface to which the solution is to be applied. One illustrative device that can be used for this purpose is described in U.S. Pat. No. 7,607,852 B2, which is hereby incorporated by reference in its entirety. Another related device that could readily adapted for application of the solution of the ascorbic acid fatty acid ester in plant oil to one or more tooth surfaces of one or more teeth of an animal in need of dental treatment is that described in U.S. Patent Application Publication No. U.S. 2011/0247162 A1, which is hereby incorporated by reference in its entirety.

Accordingly, in certain embodiments of this method of treatment or reduction of a dental condition, involving dissolution of an ascorbic acid fatty acid ester in a plant oil to one or more surfaces of one or more teeth of an animal in need thereof, the dental condition is any of dental plaque accumulation, calculus, tooth staining, halitosis, stomatitis, gingivitis, periodontitis, and combinations thereof. In a particular aspect of this embodiment, the dental condition is plaque accumulation. In other aspects of this embodiment, that animal in need thereof is a companion animal, and in even more specific aspects of this embodiment, the companion animal is a canine companion animal or a feline companion animal.

In another embodiment, the present disclosure provides a method for treating or reducing a dental condition in an animal that comprises feeding a dry food composition of the disclosure to an animal in need thereof. In one aspect of this embodiment, the method may further comprise direct application of a solution comprising an ascorbyl ester to one or more surfaces of one or more teeth of the animal in need of such treatment according to the methods describe above.

In one embodiment, the dry food composition fed to an animal in need of dental treatment or reduction in dental conditions comprises a matrix of ingredients nutritionally, organoleptically or both nutritionally and organoleptically adapted for the animal, wherein the dry food composition comprises an effective amount of an ascorbyl ester. In one aspect of this embodiment, the ascorbyl ester is an ascorbic acid fatty acid ester dissolved in a solvent, e.g., a plant oil that is applied the surface of the matrix as an outer coating layer. In another aspect, the ascorbyl ester is applied to the outer surface of the matrix as a powder. In a further aspect of this embodiment, the ascorbyl ester is applied to the outer surface of the matrix in the form of an encapsulated material. In a still further aspect, the ascorbyl ester is prepared as an encapsulated material that is incorporated within the matrix of ingredients.

In one embodiment, the dry food composition comprising a matrix of ingredients comprising an outer layer coating comprising an effective amount of an ascorbyl ester dissolved in a solvent. In certain aspects of this embodiment, the ascorbyl ester is an ascorbic acid fatty acid ester and the solvent is an oil, e.g., a plant oil. In another aspect of this embodiment, the matrix of food ingredients is formulated to meet the ordinary nutritional requirements of the animal to which the methods disclosed herein are to be applied. Such formulations can be determined by a skilled artisan based upon the animal's species, age, sex, weight, and other factors. For example, typical diets for mature canines and felines are provided in Table 1, above.

In specific embodiments, the methods of the disclosure are directed to treating or reducing a dental condition in an animal in need thereof, comprising feeding that animal a composition comprising a matrix of ingredients that are formulated with suitable sources of protein, carbohydrate, fat, and fiber, before coating. Suitable sources of protein, fat, and carbohydrate include but are not limited to those disclosed above. The matrix of ingredients may also comprise one or more additional ingredients including but not limited to the vitamins and minerals disclosed above.

In a particular aspect of this embodiment, the methods of the disclosure comprise feeding a dry food composition which comprises the matrix of ingredients that is to be coated, in which the matrix comprises, before coating, from about 10% to about 35%, from about 15% to about 30%, and from about 20% to about 25% protein. In a specific aspect, the methods of the disclosure comprise feeding a companion animal dry pet food composition in which the matrix of ingredients, before coating, comprises about 22% protein.

In another particular aspect of this embodiment, the methods of the disclosure comprise feeding a dry food composition in which the matrix comprises, before coating, from about 35% to about 75%, from about 45% to about 70%, and from about 55% to about 65% carbohydrate. In a specific aspect, the methods of the disclosure comprise feeding a composition of the disclosure, which composition is a companion animal dry pet food composition in which the matrix of ingredients, before coating, comprise about 60% carbohydrate.

In a particular aspect of this embodiment, the methods of the disclosure comprise feeding a dry food composition in which the matrix of ingredients, before coating, comprises from about 3% to about 25%, from about 5% to about 20%, from about 10% to about 15%, and from about 8% to about 14% fat. In a specific aspect, the methods of the disclosure comprise feeding a companion animal dry pet food composition in which the matrix of ingredients, before coating, comprise about 10% fat.

In a particular aspect of this embodiment, the methods of the disclosure comprise feeding a dry food composition which comprises the matrix of ingredients that is to be coated, in which the matrix of ingredients is formulated as a companion animal pet food composition, in which the matrix of ingredients comprises, before coating, from about 0.25% to about 10%, from about 0.5% to about 6%, from about 1% to about 5% crude fiber, and from about 2% to about 4% crude fiber. In a specific aspect, the methods of the disclosure comprise feeding a companion animal dry pet food composition in which the matrix of ingredients, before coating, comprises about 3% crude fiber.

In a particular aspect of this embodiment, the methods of the disclosure comprise feeding a dry food composition which comprises a matrix of ingredients that is to be coated, in which the matrix of ingredients is formulated as a companion animal pet food composition, and in which the matrix may comprise, before coating, from about 5% to about 15%, from about 6% to about 14%, from about 7% to about 13%, from about 8% to about 12%, and from about 9% to about 11% moisture. In a specific aspect, the methods of the disclosure comprise feeding a composition comprising a matrix of ingredients that, before coating, comprises about 10% moisture.

In a particular aspect of this embodiment, the methods of the disclosure comprise feeding a dry food composition which comprises a matrix of ingredients that is to be coated, in which the matrix of ingredients is formulated as a companion animal pet food composition, in which the matrix of ingredients may comprise, before coating, from about 20% protein, about 60% carbohydrate, about 13% fat, about 10% moisture and from about 2.5% to about 8.5% crude fiber.

The pet food compositions comprising the matrix of ingredients, formulated as above, are particularly suitable for feeding to canines in need of dental treatment or reduction in dental conditions. In other embodiments, pet food compositions comprising a matrix of ingredients that are particularly suitable for use in the disclosed methods of treatment or reduction of feline companion animals generally have higher levels of protein and lower levels of carbohydrate and fiber than described above.

Therefore, in another embodiment, the methods of the disclosure are directed to treating or reducing a dental condition in feline companion animal in need thereof, the method comprising feeding a dry food composition (i.e., a companion animal dry pet food composition) comprising a matrix of ingredients that, before coating, may comprise from about 20% to about 45%, from about 25% to about 40%, and from about 30% to about 35% protein. In a specific aspect, the methods of the disclosure comprise feeding a companion animal pet food composition comprising a matrix of ingredients that, before coating, comprises about 33% protein.

In a particular aspect of this embodiment, the methods of the disclosure are directed to treating or reducing a dental condition in an animal in need thereof, the method comprising feeding a feline companion animal a dry food composition in which the matrix of ingredients, before coating, comprises from about 15% to about 45%, from about 20% to about 40%, and from about 25% to about 35% carbohydrate. In a specific aspect, the methods of the disclosure comprise feeding a companion animal pet dry food composition comprising a matrix of ingredients that, before coating, comprises about 30% carbohydrate.

In a particular aspect of this embodiment, the methods of the disclosure are directed to treating or reducing a dental condition in an animal in need thereof, the method comprising feeding a feline companion animal a dry food composition in which the matrix of ingredients, before coating, comprises from about 5% to about 35%, from about 10% to about 30%, and from about 15% to about 25% fat. In a specific aspect, the methods of the disclosure comprise feeding a companion animal pet dry food composition comprising a matrix of ingredients that, before coating, comprises about 20% fat.

In a particular aspect of this embodiment, the methods of the disclosure are directed to treating or reducing a dental condition in an animal in need thereof, the method comprising feeding a feline companion animal a dry food composition in which the matrix of ingredients, before coating, comprises from about 4% to about 15%, from about 5% to about 12%, from about 6% to about 10%, and from about 7% to about 9% crude fiber. In a specific aspect, the methods of the disclosure comprise feeding a companion animal pet dry food composition comprising a matrix of ingredients that, before coating, comprises about 8% crude fiber.

In a particular aspect of this embodiment, the methods of the disclosure are directed to treating or reducing a dental condition in an animal in need of such treatment, the method comprising feeding a feline companion animal a dry composition in which the matrix of ingredients, before coating, comprises about 33% protein, about 30% carbohydrate, about 20% fat, about 8% crude fiber and about 10% moisture.

In one illustrative example, the matrix of ingredients, before coating, may comprise inter alia, vitamins, minerals and other additives. In one aspect of this embodiment, the matrix of ingredients of an illustrative pet food may comprise, before coating, chicken corn meal, ground grain sorghum, ground wheat, chicken by-product meal, brewers rice, soybean meal, animal fat, (preserved with BHA and propyl gallate), natural flavor, vegetable oil, dried egg product, flaxseed, preserved with BHT and BHA, beta-carotene, minerals (iodized salt, calcium carbonate, dicalcium phosphate, potassium chloride, ferrous sulfate, zinc oxide, copper sulfate, manganous oxide, calcium iodate, sodium selenite), and vitamins (choline chloride, vitamin A supplement, vitamin D₃ supplement, vitamin E supplement, niacin, thiamine, calcium pantothenate, pyridoxine hydrochloride, riboflavin, folic acid, biotin, vitamin B₁₂ supplement).

In particular embodiments, the matrix of ingredients of the compositions are prepared in a dry form using conventional processes. In one contemplated embodiment, dry ingredients, including, for example, animal protein sources, plant protein sources, grains, etc., are ground and mixed together. Moist or liquid ingredients, including fats, oils, animal protein sources, water, etc., are then added to and mixed with the dry mix. The mixture is then processed into kibbles or similar dry pieces. Kibble is often formed using an extrusion process in which the mixture of dry and wet ingredients is subjected to mechanical work at a high pressure and temperature, and forced through small openings and cut off into kibble by a rotating knife. The wet kibble is then dried and optionally coated, e.g., with an inner coating layer, before coating with the solution of an ascorbic acid fatty acid ester in a plant oil. Kibble also can be made from the dough using a baking process, rather than extrusion, wherein the dough is placed into a mold before dry-heat processing. Kibble also can be made from a food matrix undergoing pelletization.

Treats of the present invention can be prepared by, for example, an extrusion or baking process similar to those described above for dry food to provide the dry compositions that are then optionally coated with an inner coating layer, before coating with the solution of an ascorbic acid fatty acid ester in a plant oil.

In another embodiment, the matrix of ingredients that is optionally coated with an inner coating layer before coating with the solution of an ascorbyl ester in a solvent, e.g., an ascorbic acid fatty acid ester, such as ascorbyl palmitate, in an oil, e.g., a plant oil such as soybean oil, are prepared as a food supplement. Supplements include, for example, a feed or pet food used with another feed or pet food to improve the nutritive balance or performance of the total. The contemplated supplements include compositions that are fed undiluted as a supplement to other feeds or pet foods, offered free choice with other parts of an animal's ration that are separately available, or diluted and mixed with an animal's regular feed or pet food to produce a complete feed or pet food.

In another embodiment, the matrix of ingredients that is optionally coated with an inner coating layer, before coating with the solution of an ascorbyl ester in a solvent, e.g., an ascorbic acid fatty acid ester, such as ascorbyl palmitate, in an oil, e.g., a plant oil such as soybean oil, is prepared as a treat. Treats include, for example, compositions that are given to an animal to entice the animal to eat during a non-meal time. Contemplated treats for canines include, for example, dog biscuits in the shape of dog bones. Treats can be nutritional, wherein the composition comprises one or more nutrients, and can, for example, have a composition as described above for food. Non-nutritional treats encompass any other treats that are non-toxic. In each instance, the dry material is coated with the solution of an ascorbic acid fatty acid ester in a plant oil. In certain aspects, the treat further comprises an optional inner coating layer disposed between the outer surface of the treat and the outer coating layer of the solution of an ascorbyl ester in a solvent, e.g., an ascorbic acid fatty acid ester, such as ascorbyl palmitate, in an oil, e.g., a plant oil such as soybean oil.

In certain embodiments, the matrix of ingredients of the compositions used in the methods of the present disclosure, whether a companion animal pet food composition, dietary supplement, or treat may further comprise at least one of a commercial liquid palatant enhancer or other flavor composition to create a novel flavor palatant which can then be included in the matrix formulation. Suitable commercial liquid palatant enhancers for use with compositions used in the disclosed methods can include any known or commercially available liquid palatant enhancers commercially available from pet food palatant enhancer or other flavor suppliers known to those of skill in the art.

The compositions that are used in the disclosed methods may comprise a physically discrete matrix of ingredients which include kibbles as well as, in other embodiments, bits and other discrete materials, such as a solid treat, a supplement or the like, which are coated with an outer coating layer applied as a solution of ascorbic acid fatty acid ester in a plant oil, and which may further comprise an inner coating layer disposed on the surface of the matrix before application of the outer coating layer. Again, by coating the surface is meant that at least enough of the surface of the matrix of ingredients is covered with the film so as to achieve the desired effect. The entire surface need not be coated. For example, in certain embodiments, an effective amount of the ascorbic acid fatty acid ester can be delivered by feeding food compositions comprising a matrix of ingredients, in which about 5%, about 10%, about 15%, about 20%, about 25%, about 30%, about 40%, about 45%, about 50%, about 60%, about 70%, about 80%, about 90%, or up to about 100% of the surface of the matrix has been coated with either or both of the outer coating layer and, when present, the inner coating layer.

The dry pet food compositions used in the dental treatment methods of the disclosure comprise a matrix comprising an outer coating layer. The process by which the outer coating layer, as well as the inner coating layer (when present) is applied to the matrix of ingredients (or the matrix of ingredients carrying the inner coating layer) employ any of the common procedures known to be effective in applying films to objects. These procedures include casting, spraying, grafting, sputtering, flowing, calendaring, and the like. It can be done to the matrix prior to being cut into physically discrete portions or, preferably, after the physically discrete matrix particles are formed, thus ensuring an even distribution on the entire matrix, if so desired.

In certain embodiments, the dry pet food compositions used in the dental treatment methods of the disclosure comprise a matrix comprising an outer coating layer as well as an inner coating layer. In certain embodiments, the inner coating layer comprises a polymer which can be any of zein, casein, starch, cellulose, gum, gelatin, starch/synthetic polymer, e.g, starch/low density polyethylene, and the like, as well as combinations thereof. In certain aspects of this embodiment, the inner coating layer can be from about 1 to about 2000 microns thick, or from about 2 to about 1000 microns. The composition and thickness of the inner coating layer are chosen to provide the desired degree of inhibition of absorption of the outer coating layer into the matrix.

In certain aspects of this embodiment, an aqueous solution, sol, or suspension is applied to the surface of the matrix of ingredients to form the inner coating layer that may comprise monosaccharides such as glucose, fructose, mannose, arabinose; disaccharides and trisaccharides such as sucrose, lactose, maltose, trehalose, lactulose; corn and rice syrup solids; dextrins such a corn, wheat, rice and tapioca dextrins; maltodextrins; starches such as rice, wheat, corn, potato, tapioca starches, or these starches modified by chemical modification. In other aspects, the aqueous solution, sol, or suspension that is applied to the surface of the matrix to form the inner coating layer may comprise oligosaccharides such as fructooligosccharides, alginates, chitosans; gums such as carrageen, and gum arabic; polyols such as glycerol, sorbitol, mannitol, xylitol, erythritol; esters of polyols such as sucrose esters, polyglycol esters, glycerol esters, polyglycerol esters, sorbitan esters; sorbitol; molasses; honey; gelatins; peptides; proteins and modified proteins such as whey liquid, whey powder, whey concentrate, whey isolate, whey protein isolate, or a high lactose whey by-product. In other aspects of this embodiment, the aqueous solution, sol, or suspension that is applied to the surface of the matrix of ingredients to form the inner coating layer may comprise meat broth solids such as chicken broth, chicken broth solids, soy protein, and egg white.

In still further aspects of this embodiment, the aqueous solution, sol, or suspension that is applied to the surface of the matrix of ingredients to form the inner coating layer may comprise a palatant. Palatants useful for this purpose are palatability enhancers which are typically employed to enhance the overall palatability of the composition and potentially to overcome any negative flavor effects arising from other components of the composition. Palatants particularly useful in accordance with the invention are those described in US2003/0086961, which is hereby incorporated by reference. Particularly useful are protein hydrolysates which typically provide a savory taste attractive to various animals including cats and dogs. The palatants include animal digest; animal hydrolysates; animal internal organs such as liver, lungs and heart; meats such as lamb, beef, pork, chicken and turkey; seafoods such as fish, crab, and shrimp; dairy products such as milk and cheese; yeasts; peptides; amino acids; nucleotides; fat; oil; artificial meat and/or seafood flavors; mallard reactants; sugars; plant extracts and other aromas nature and/or artificial that are attractive to the animals. For cats, preferred palatants include chicken liver hydrolysates and fish-based hydrolysates; for dogs, a protein digest or hydrolysate is particularly useful.

In other aspects of this embodiment, the aqueous solution, sol, or suspension that is applied to the surface of the matrix of ingredients to form the inner coating layer may comprise combinations of two or more of any of these materials.

In another aspect of this embodiment, the dry pet food compositions used in the dental treatment methods of the disclosure comprise a matrix comprising an inner coating layer that may be prepared as an aqueous solution, sol, or suspension that is applied to the surface of the matrix using any of the methods disclosed above as well as any known in the art. In certain aspects of these embodiments the amount of aqueous material applied to the matrix represents from about 1% to about 20%, from about 2% to about 15%, or from about 5 to about 10% of the weight of the matrix. The coated matrix of ingredients is, generally, then dried before application of the outer coating layer. In one aspect of this embodiment, the matrix of ingredients to which the inner coating layer has been applied is placed in a tumbling drum to facilitate uniform distribution of the inner coating layer. In certain instances the matrix of ingredients in the tumbling drum can be exposed to heated air to for drying, e.g., reducing the moisture content of the coated matrix of ingredients to a level of about 10%. Accordingly, after drying, the inner coating layer represents from about 0.025% to about 10%, from about 0.05% to about 7.5%, from about 0.1% to about 5.0%, from about 0.2% to about 2.5%, or from about 0.25% to about 1.25% of the weight of the matrix of ingredients on a dry matter basis.

The inner coating layer, in certain embodiments inhibits absorption of components of the outer coating layer into the matrix of ingredients, thereby maximizing the amount of ascorbic acid fatty acid ester and plant oil solvent disposed on the surface of the coated matrix of ingredients.

In certain embodiments, the dry pet food compositions used in the dental treatment methods of the disclosure comprise a matrix comprising an outer coating layer that can be prepared by dissolving the ascorbic acid fatty acid ester in a plant oil and applying that solution to the matrix of ingredients that, in certain embodiments, may already comprise an inner coating layer. The specific combination of fatty acid for esterification of ascorbic acid and the plant oil solvent used will depend on the solubility of the former in the latter, the acceptability of the taste of the solution to the animal to be treated, as well as the residence time of the solution on the teeth of the treated animal. Each of these parameters can be readily determined.

In one aspect of this embodiment, the plant oil is selected from among corn oil, cottonseed oil, canola oil, rapeseed oil, olive oil, palm oil, palm kernel oil, peanut oil, safflower oil, sesame oil, soybean oil, sunflower oil, wheat germ oil, almond oil, apricot oil, argan oil, avocado oil, ben oil, cashew oil, castor oil, grape seed oil, hazelnut oil, hemp seed oil, linseed oil, mustard oil, neem oil, pumpkin seed oil, rice bran oil, and combinations thereof. In a specific aspect the plant oil is soybean oil.

In another aspect of this embodiment, the fatty acid that is esterified to ascorbic acid is any of lauric acid, myristic acid, palmitic acid, stearic acid, and arachidic acid. In a specific aspect, the esterified fatty acid is palmitic acid, i.e., the ascorbic acid fatty acid ester is ascorbyl palmitate.

In certain embodiments, the outer coating layer comprises an ascorbic acid fatty acid ester dissolved in a plant oil. In another embodiment, the outer coating layer consists essentially of an ascorbic acid fatty acid ester dissolved in a plant oil, and, in a specific aspect of this embodiment, the ascorbic acid fatty acid ester is the only active agent in the solution applied to the matrix to form the outer coating layer. In a still further embodiment, the outer coating layer consists of an ascorbic acid fatty acid ester dissolved in a plant oil.

In a specific embodiment, the outer coating layer comprises ascorbyl palmitate dissolved in soybean oil. In another embodiment, the outer coating layer consists essentially of ascorbyl palmitate dissolved in soybean oil and, in a specific aspect of this embodiment, ascorbyl palmitate is the only active agent in the solution applied to the matrix to form the outer coating layer. In a still further embodiment, the outer coating layer consists of ascorbyl palmitate dissolved in soybean oil.

The process by which the outer coating layer is applied to the matrix of ingredients (or matrix of ingredients carrying the inner coating layer) may employ any of the common procedures known to be effective in applying films to objects as well as commercially-available equipment useful for such purposes. These procedures include, but are not limited to, casting, spraying, grafting, sputtering, flowing, calendaring, and the like. Application of the outer coating layer to the matrix may be carried out before the formulations, e.g., the cooked and extruded material is cut into physically discrete portions or, preferably, after the physically discrete matrix of ingredients is formed, thus ensuring an even distribution on the entire matrix of ingredients. In one aspect of this embodiment, the matrix of ingredients to which the outer coating layer has been applied is placed in a tumbling drum to facilitate uniform distribution of the solution of the outer coating layer over the matrix.

In one embodiment, the outer coating layer represents from about 0.025% to about 10%, from about 0.05% to about 7.5%, from about 0.1% to about 5.0%, from about 0.2% to about 2.5%, or from about 0.25% to about 1.25% of the weight of the coated matrix on a (w/w) basis. Accordingly, the ascorbic acid fatty acid ester of the outer coating layer can represent 0.005% to about 5%, from about 0.010% to about 2.5%, from about 0.025% to about 1.0%, from about 0.050% to about 0.5%, of the weight of the coated matrix of ingredients, on a dry matter basis. In a specific aspect of this embodiment, the ascorbic acid fatty acid ester is ascorbyl palmitate and it represents about 0.1% of the weight of the matrix of ingredients on a dry weight basis.

Accordingly, in certain embodiments, the disclosure provides a method for treating or reducing a dental condition in an animal comprising feeding the animal a dry food composition comprising (i) a matrix of ingredients nutritionally or organoleptically adapted for the animal and (ii) an outer coating layer, wherein the outer coating layer is applied to the matrix as a solution comprising an effective amount of an ascorbyl ester dissolved in a solvent. In one aspect of this embodiment, the ascorbyl ester is an ascorbic acid fatty acid ester, e.g., ascorbyl palmitate. In another aspect of this embodiment, the solvent is an oil, e.g., a plant oil such soybean oil. In other specific aspects of this embodiment, the dental condition to be treated or reduced is any of dental plaque accumulation, calculus, tooth staining, halitosis, stomatitis, gingivitis, periodontitis, and combinations thereof. In a particular aspect of this embodiment, the dental condition is dental plaque accumulation. In other aspects of this embodiment, that animal in need of treatment is a companion animal, and in even more specific aspects of this embodiment, the companion animal is a canine or a feline.

### EXAMPLES

### Example 1

### Inhibition of Dental Plaque Formation with Ascorbyl Palmitate by Feeding Compositions of the Disclosure

Ascorbyl palmitate was assessed for its ability to inhibit dental plaque formation when applied indirectly, i.e., by feeding animals a pet food composition as described. In this experiment, the 8-day GCPI dental plaques quantification model of Scherl et al. was used (Scherl et al. (2007) J. Vet. Dent. 24(1): 14-20). The teeth of the test animals are cleaned at the beginning of the study, after which the animals were fed either a control food (a commercially-available canine pet food composition) or the same food upon which a solution of ascorbyl palmitate in soybean oil was sprayed to a final concentration (w/w) of 0.1% of the total weight of the food. The food coated with the ascorbyl palmitate solution was fed to dogs over a period of four days and dental plaque accumulation was quantified and compared with their dental plaque accumulation while on the control food.

Plaque accumulation in the control sample had an average score of 68.2±11.9 while the score determined for dogs treated with ascorbyl palmitate was 53.7±12.1, representing a 21.3% improvement. The GCPI uses a scale of 0-100%.

The data obtained in this experiment demonstrate that dogs fed a food comprising ascorbyl palmitate inhibited plaque formation.

### Example 2

### Inhibition of Dental Plaque Formation with Ascorbyl Palmitate—Direct Application

Ascorbyl palmitate was assessed for its ability to inhibit dental plaque in 24-hour plaque accumulation model, according to a modification of the method of Scherl et al. (Scherl et al. (2007) J. Vet. Dent. 24(1): 14-20), in which the compound to be tested is directly applied to the teeth as a solution using a dental swab as applicator, and the dental plaque accumulation time was set at 24 hours.

A solution of ascorbyl palmitate (0.3 g in 10 mL soybean oil) was administered to the cleaned buccal tooth and gingival surfaces of each dog using a dental swab, in such a manner that 0.3 g of ascorbyl palmitate was applied. Control animals were treated with soybean oil alone. Twenty-four hours after application, the accumulation of dental plaque was quantitatively evaluated using a standardized method designated as the gingival contour plaque index (“GCPI”). Plaque accumulation in the control sample had an average score of 24.9±9.4 while that determined for dogs treated with ascorbyl palmitate was 18.9±9, representing a 27.8% improvement. The GCPI uses a scale of 0-100%.

The data obtained in this experiment demonstrate that direct application of a solution of ascorbyl palmitate dissolved in soybean oil to the animal's teeth inhibited plaque formation.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material. Except to the extent stated otherwise, all percentages used in this specification are weight percentages on a dry matter basis. The phrase “dry matter basis” means the component concentration in the composition after any moisture in the composition is removed.

1. A pet food composition for treating or reducing a dental condition in an animal in need thereof, wherein the pet food composition is a dry food composition comprising (i) a matrix of ingredients nutritionally or organoleptically adapted for the animal and (ii) an outer coating layer, wherein the outer coating layer is applied to the matrix as a solution comprising an effective amount of an ascorbic acid fatty acid ester dissolved in a plant oil.

2. The pet food composition of claim 1, wherein the esterified fatty acid is selected from the group consisting of lauric acid, myristic acid, palmitic acid, stearic acid, and arachidic acid.

3. The pet food composition of claim 2, wherein the esterified fatty acid is palmitic acid.

4. The pet food composition of claim 1, wherein the plant oil is selected from the group consisting of corn oil, cottonseed oil, canola oil, rapeseed oil, olive oil, palm oil, palm kernel oil, peanut oil, safflower oil, sesame oil, soybean oil, sunflower oil, wheat germ oil, almond oil, apricot oil, argan oil, avocado oil, ben oil, cashew oil, castor oil, grape seed oil, hazelnut oil, hemp seed oil, linseed oil, mustard oil, neem oil, pumpkin seed oil, rice bran oil, and combinations thereof.

5. The pet food composition of claim 4, wherein the plant oil is soybean oil.

6. The pet food composition of claim 1, wherein the solution comprises from about 5% to about 50% (w/w) ascorbic acid fatty acid ester.

7. (canceled)

8. The pet food composition of claim 1, wherein the animal is a companion animal.

9. The pet food composition of claim 8, wherein the companion animal is a canine or a feline.

10. The pet food composition of claim 1, wherein the dental condition is selected from the group consisting of dental plaque accumulation, calculus, tooth staining, halitosis, stomatitis, gingivitis, periodontitis, and combinations thereof.

11. The pet food composition of claim 10, wherein the dental condition is dental plaque accumulation.

12. The pet food composition of claim 1, wherein the matrix further comprises an inner coating layer disposed between the matrix and the outer coating layer, wherein the inner coating layer is applied to the surface of the matrix, and wherein the inner coating layer inhibits absorption of the outer coating layer into the matrix

13. A method for treating or reducing a dental condition in an animal, the method comprising feeding the animal a dry food composition comprising (i) a matrix of ingredients nutritionally or organoleptically adapted for the animal and (ii) an outer coating layer, wherein the outer coating layer is applied to the matrix as a solution comprising an effective amount of an ascorbic acid fatty acid ester dissolved in a plant oil.

14. The method of claim 13, wherein the matrix further comprises an inner coating layer disposed between the matrix and the outer coating layer, wherein the inner coating layer is applied to the surface of the matrix, and wherein the inner coating layer inhibits absorption of the outer coating layer into the matrix.

15. The method of claim 13, wherein the dental condition is selected from the group consisting of dental plaque accumulation, calculus, tooth staining, halitosis, stomatitis, gingivitis, periodontitis, and combinations thereof.

16. The method of claim 15, wherein the dental condition is dental plaque accumulation.

17. The method of claim 13, wherein the ascorbic acid fatty acid ester is selected from the group consisting of lauric acid, myristic acid, palmitic acid, stearic acid, and arachidic acid.

18. The method of claim 17, wherein the ascorbic acid fatty acid ester is palmitic acid.

19. The method of claim 13, wherein the plant oil is selected from the group consisting of corn oil, cottonseed oil, canola oil, rapeseed oil, olive oil, palm oil, palm kernel oil, peanut oil, safflower oil, sesame oil, soybean oil, sunflower oil, wheat germ oil, almond oil, apricot oil, argan oil, avocado oil, ben oil, cashew oil, castor oil, grape seed oil, hazelnut oil, hemp seed oil, linseed oil, mustard oil, neem oil, pumpkin seed oil, rice bran oil, and combinations thereof.

20. The method of claim 19, wherein the plant oil is soybean oil.

21. (canceled)

22. (canceled)

23. The method of claim 13, wherein the animal is a companion animal.

24. The method of claim 23, wherein the companion animal is a canine or feline.

25. A method for treating or reducing a dental condition in an animal, the method comprising applying to one or more surfaces of one or more teeth of an animal in need of such treatment an effective amount of a solution comprising an ascorbic acid fatty acid ester dissolved in a plant oil.

26. The method of claim 25, wherein the dental condition is selected from the group consisting of dental plaque accumulation, calculus, tooth staining, halitosis, stomatitis, gingivitis, periodontitis, and combinations thereof.

27. The method of claim 26, wherein the dental condition is dental plaque accumulation.

28. The method of claim 25, wherein the ascorbic acid fatty acid ester is selected from the group consisting of lauric acid, myristic acid, palmitic acid, stearic acid, and arachidic acid.

29. The method of claim 28, wherein the ascorbic acid fatty acid ester is palmitic acid.

30. The method of claim 25, wherein the plant oil is selected from the group consisting of corn oil, cottonseed oil, canola oil, rapeseed oil, olive oil, palm oil, palm kernel oil, peanut oil, safflower oil, sesame oil, soybean oil, sunflower oil, wheat germ oil, almond oil, apricot oil, argan oil, avocado oil, ben oil, cashew oil, castor oil, grape seed oil, hazelnut oil, hemp seed oil, linseed oil, mustard oil, neem oil, pumpkin seed oil, rice bran oil, and combinations thereof.

31. The method of claim 30, wherein the plant oil is soybean oil.

32. The method of claim 25, wherein the solution comprises from about 5% to about 50% (w/w) ascorbic acid fatty acid ester.

33. (canceled)

34. (canceled)

35. The method of claim 25, wherein the animal is a companion animal.

36. The method of claim 35, wherein the companion animal is a canine or a feline.

37. The method of claim 25, wherein the solution is applied with a liquid applicator selected from the group consisting of a dental swab, a towelette, a sprayer, a sponge, a brush.

38. The method of claim 25, wherein the solution is applied with a liquids applicator device, said device comprising an elongated hollow cartridge housing, a cartridge assembly disposable within the cartridge housing, an applicator head portion removably attached to an open end of the cartridge housing, and a liquids dispensing mechanism that causes the solution to be dispensed from the cartridge into the applicator head portion.
